(19) United States
(12) Patent Application Publication
(10) Pub. No.: US 2008/0319079 A1
(43) Pub. Date: Dec. 25, 2008

(54) Method for Administering Formoterol Using a Nebulizer

(76) Inventor: Stephen J. Feanny, Vaughan (CA)

Correspondence Address: OSLER, HOSKIN & HARCOURT LLP (OSLER OTTAWA), 1000 DE LA GAUCHETIERE STREET WEST, SUITE 2100, MONTREAL, QC, H3B-4W5, CA

(21) Appl. No.: 12/094,947

(22) PCT Filed: Nov. 23, 2006
(86) PCT No.: PCT/CA06/01920

§ 371 Date: May 23, 2008

(51) Int. Cl. A61K 31/194; A61K 31/135; A61P 11/00

(52) U.S. Cl. 514/574; 514/653

(57) ABSTRACT

The present invention provides a method of administering formoterol, for example as a fumarate dihydrate, using a nebulizer device for the treatment of bronchoconstriction, such as that caused by asthma and chronic obstructive pulmonary disease. The method of this invention is particularly useful in pediatric, geriatric, handicapped and acutely ill individuals who have been denied the benefits of this medication due to the standard use of dry powder inhalers. The present invention addresses the difficulty of making available a viable commercial nebulizer solution for these individuals who cannot optimally use powder inhaler devices, which are the only presently available delivery systems for this medication.

### CROSS REFERENCE TO RELATED APPLICATION

The present application claims priority to U.S. provisional patent application Ser. No. 60/739,027, filed Nov. 23, 2005, which is incorporated herein by reference.

### FIELD OF THE INVENTION

The present invention pertains to the field of nebulization and, in particular, to the field of nebulization of formoterol.

### BACKGROUND

Presently there are several effective drugs for the treatment of bronchospasm in patients with asthma and related respiratory illnesses such as COPD (Chronic obstructive pulmonary disease). Many of the products are short acting in duration (salbutamol, terbutaline, albuterol) lasting four to six hours.

Salmeterol is long acting lasting twelve hours but is not appropriate for acute relief as its onset of action is too long being approximately thirty minutes in contrast to the above routinely used short active Beta-2 receptor agonists/bronchodilators which have an onset of action of approximately three minutes.

Emergency room facilities of hospitals worldwide continue to use short acting bronchodilators daily for the thousands of acutely ill asthmatic patients who visit their facilities. This provides temporary relief (4 to 6 hours) while waiting for anti-inflammatory agents such as corticosteroids to take effect and often results in repeat same day visits or hospitalization as the effect of the short acting bronchodilators wears off before the anti-inflammatory medication can take effect. Scientific advances have led to the recognition of inflammation as the primary pathology in asthma and the resultant availability of inhaled corticosteroids as the mainstay of therapy. More recently the use of inhaled corticosteroids in synergy with long acting β2 agonists has become the gold standard of treatment. The preparations containing these substances are delivered by inhaler devices. The patient groups which have not benefited from the use of inhaled long acting β₂-agonists include pediatric, geriatric, handicapped and acutely individuals who experience difficulty using such inhaler devices.

Formoterol (N-[2-hydroxy-5-(1-hydroxy-2-((2-(4-methoxyphenyl)-1-methylethyl)-amino)-ethyl)phenyl]formamide) is an anilide of Formula I derived from adrenaline and is used as a β₂ receptor agonist in inhalation therapy of respiratory diseases, such as bronchial asthma and COPD (Chronic obstructive pulmonary disease). In patients with reversible obstructive respiratory diseases, formoterol has a bronchodilatory effect. Only 1-3 minutes after inhalation the effect sets in and the bronchodilatory effect is still significantly present after 12 hours. Formoterol inhibits the release of leukotrienes and other messenger substances involved with inflammation. In addition, formoterol may bring about a hyperglycaemic activity. Formoterol has been shown to have no more side effects than short acting bronchodilators when used in repeated doses administered frequently up to a maximum dose of 80 μg (microgram) per day.

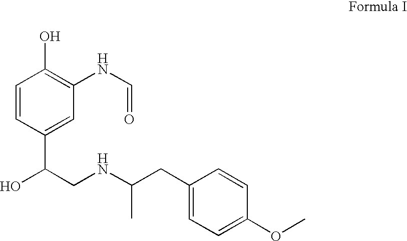

Formoterol is ideal for use in treating acute asthma because of its rapid onset of action, long duration of activity and its ability to be used safely in repeated doses. These characteristics allow the administering physician the added luxury of knowing that patients treated will have bronchodilation for up to 12 hours, which is approximately 3 times that of salbutamol and terbutaline, the most commonly used bronchodilators. The safety and efficiency of formoterol has been well documented in numerous studies.

Additionally formoterol has been shown to have anti-inflammatory activity reducing cells (eosinophils) and substances (cytokines including leukotrienes) that are present in the airways of asthmatic patients. This is a characteristic not shown with the commonly used short acting bronchodilators.

Most often formoterol is formulated as a dry powder and administered via devices such as the Turbuhaler® and the Aerolizer®. See, e.g., Seberova et al. (2000) Respir. Med. 94(6):607-611; Lotvall et al. (1999) Can. Respir. J. 6(5):412-416; Campbell et al. (1999) Respir. Med. 93(4):236-244; Nightingale et al. (1999) Am. J. Respir. Crit. Care Med. 159(6):1786-1790; Lecaillon et al. (1999) Eur. J. Clin. Pharmacol. 55(2):131-138; Bartow et al. (1998) Drugs 55(2):303-322; Ekstrom et al. (1998) Respir. Med. 92(8):1040-1045; Ringdal et al. (1998) Respir. Med. 92(8):1017-1021; Totterman et al. (1998) Eur. Respir. J. 12(3):573-579; Palmquist et al. (1997) Eur. Respir. J. 10(11):2484-2489; Nielsen et al. (1997) Eur. Respir. J. 10(9):2105-2109; Ullman et al. (1996) Allergy 51(10):745-748; Selroos et al. (1996) Clin. Immunother. 6:273-299; and Schreurs et al. (1996) Eur. Respir. J. 9(8):1678-1683.

Formoterol is also available as a tablet and a dry syrup in certain areas of the world (e.g., Atock®, marketed by Yamanouchi Pharmaceutical Co. Ltd., Japan). Formoterol formulations are also available in other areas (e.g., Europe and U.S.) for propellant-based metered dose inhalers and dry powder inhalers (e.g., Turbuhaler®, Aerolizer® and Foradil Aerolizer®). None of these formulations are water based. Sterile, stable, aqueous based inhalation solutions of formoterol for direct nebulization are not available, nor have they been reported.

Other compositions containing formoterol have been disclosed in U.S. Pat. Nos. 5,677,809, 6,126,919, 5,733,526, 6,071,971, 6,068,833, 5,795,564, 6,040,344, 6,041,777, 5,874,481, 5,965,622 and 6,161,536.

The production and use of dry powder formulations of formoterol for use in dry powder inhaler devices (Foradil inhaler and Oxis turbuhaler) as well as in combination dry powder inhaler devices (Symbicort—formoterol and budesonide—U.S. 2001049396) are known. However, the best device for administering bronchodilators for the acute asthma is a nebulizer. The nebulizer has been shown to deposit up to 80% of administered drug to the small airways where their action occurs compared to a maximum of 60% with other devices, including dry powder inhalers.

In the past it has been found that liquid aerosol formulations of formoterol are not suitable for use in inhalers intended for ambulatory inhalation treatment since formoterol cannot be stored in a sufficiently stable manner in solution to guarantee the pharmaceutical quality of the formulation over lengthy periods of time. For this reason, formoterol has previously only been used in powder form for inhalation therapy.

U.S. Pat. No. 6,150,418 discloses a “liquid active substance concentrate” containing formoterol in the form of its free base or in the form of one of the pharmacologically acceptable salts or addition products (adducts) thereof as active substance. This “liquid active substance concentrate” is reported to be a concentrated (i.e., greater than 10 mg/ml, preferably 75 to 500 mg/ml) solution or suspension that is stable for a period of several months possibly up to several years without any deterioration in the pharmaceutical quality. This patent teaches that it is the high concentration that allows for the stability of the concentrate. The “liquid active substance concentrate” is not suitable for direct administration to a patient.

U.S. Pat. No. 6,040,344 teaches that an aqueous aerosol formulation for use in a nebulizer may be prepared by dissolving formoterol tartrate in citrate buffered saline, buffered to pH 5. Because of the problematic stability of R,R-formoterol L-tartrate in aqueous solution, this formulation was not found to be attractive for long term storage.

The solvent used, pH of the solution and temperature have all been shown to affect the pharmacologically active shelf life of formoterol-containing preparations (International PCT Application No. WO 02/083079).

Powdered formulations given by inhalers are more difficult to administer particularly to the young, the elderly and the acutely ill who are most often the patients in need of such therapy. Hence there is a need for formulations of compounds such as formoterol, in a form that can be stored safely for long periods and used effectively and easily. Formoterol-containing powder formulations are known to be stable (in terms of pharmacological activity) for up to 2 years when stored at room temperature.

There remains a need for an effective and convenient method of administering formoterol, or a salt or adduct thereof, using a stable formulation of formoterol.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a method for administering formoterol using a nebulizer. In accordance with one aspect of the present invention, there is provided a method for the treatment, prevention, or amelioration of one or more symptoms of bronchoconstrictive disorders in a patient, comprising the steps of: providing a micronized powder formulation comprising formoterol, or a pharmaceutically acceptable salt or adduct thereof, and a pharmaceutically acceptable excipient; dissolving the powder formulation in a solvent to form a nebulizer solution; and administering an amount of said nebulizer solution to said patient via a nebulizer within 30 minutes following the dissolving step.

In accordance with another aspect of the invention, there is provided a kit for administering formoterol using a nebulizer, comprising: one or more containers of a micronized powder formulation comprising said formoterol, or a pharmaceutically acceptable salt or adduct thereof, and a pharmaceutically acceptable excipient; and instructions for dissolving said powder formulation with a solvent and administering the resulting nebulizer solution to a patient within 30 minutes of mixing.

In accordance with another aspect of the invention, there is provided a use of a micronized powder formulation comprising formoterol, or a pharmaceutically acceptable salt or adduct thereof, and a pharmaceutically acceptable excipient, for the treatment, prevention, or amelioration of one or more symptoms of bronchoconstrictive disorders, wherein said powder formulation is suitable for dissolution in a solvent for administration via a nebulizer within 30 minutes following mixture.

The method and use of the present invention are particularly useful for the treatment of patients (e.g., pediatric, geriatric, handicapped and acutely ill) who are presently omitted from what is accepted as the gold standard of care.

### BRIEF DESCRIPTION OF THE FIGURES

FIG. 1A depicts the dissolution step according to one embodiment of the method of the present invention in which the formoterol powder formulation is released as a unit dose from a blister pack directly into the nebulizer receptacle and the nebulizer is one that makes use of compressed oxygen/air from an in-house (e.g., hospital) source. FIG. 1B is a cross-section of a blister pack having blisters each containing a unit dose of the formoterol powder formulation.

FIG. 2 depicts the dissolution step according to one embodiment of the method of the present invention in which the formoterol powder formulation is released as a unit dose from a blister pack directly into the nebulizer receptacle (reservoir) and the nebulizer is a home nebulizer.

FIG. 3 depicts administration of a nebulizer solution during patient therapy.

### DETAILED DESCRIPTION OF THE INVENTION

The present invention pertains to a method of administering formoterol, or a pharmaceutically acceptable salt or adduct thereof, to a patient by mixing a micronized powder formulation comprising formoterol, or a pharmaceutically acceptable salt or adduct thereof, and a pharmaceutically acceptable excipient, with a solvent, such as an aqueous diluent, to form a nebulizer solution, and administering the resulting solution to the patient via a nebulizer within 30 minutes following the dissolving step. In accordance with a specific embodiment of the present invention, the resulting solution is administered to the patient within 15 minutes following the dissolving step. By administering the resulting solution within 30 minutes, or 15 minutes, following the mixing step, the effect of formoterol degradation in the presence of water is avoided or minimized. This method thus addresses the difficulty of producing a viable commercial nebulizer solution of formoterol and permits the use of formoterol as a fine powder directly in a solution for immediate administration using a nebulizer device. As used herein, the term “immediate administration”, refers to administration within approximately 30 minutes following dissolution of the formoterol powder formulation in a solvent, such as an aqueous diluent.

### Powder Formulation

In accordance with the present invention the micronized powder formulation of formoterol includes formoterol in the form of its free base, or in the form of one of the pharmacologically acceptable salts or addition products (adducts) thereof, as active substance. The term “formoterol” as used herein is intended to encompass formoterol free base according to Formula I as well as any pharmaceutically acceptable salt or adduct thereof, unless otherwise specified or clearly stated in the context. The preferred salt is formoterol fumarate, whilst the preferred addition product, or adduct, is a hydrate of formoterol.

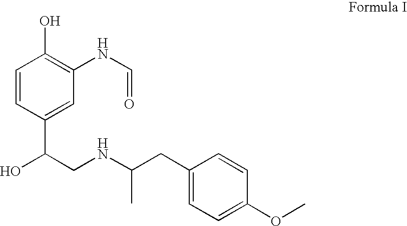

Formoterol is a compound that can exist in several stereochemical forms. The present invention includes the use of individual stereoisomers and mixtures thereof. It is intended that this invention includes the use of geometrical isomers, rotational isomers, racemates, diastereomers and enantiomers, in particular the R,R enantiomer of formoterol.

As used herein suitable physiologic salts of formoterol include but are not restricted to acid addition salts derived from inorganic and organic acids such as the hydrochloride, hydrobromide, sulfate, phosphate, maleate, fumarate, tartrate, citrate, benzoate, 4-methoxybenzoate, 2- or 4-hydroxybenzoate, 4-chlorobenzoate, p-toluenesulphonate, methanesulphonate, ascorbate, salicylate, acetate, succinate, lactate, glutarate, gluconate, tricarballylate, hydroxyl-naphthalene-carboxylate or oleate. Formoterol is preferably used in the form of its fumarate salt and as a dihydrate of this salt.

The method of the present invention can be used to administer formoterol as the sole active ingredient. Alternatively, the method of the present invention can be used to administer formoterol in combination with at least one other active ingredient. Combination therapies and compositions containing formoterol in combination with other active ingredients are known. For example, U.S. Pat. Nos. 6,004,537, 5,972,919 and 5,674,860 disclose the combination of formoterol and budenoside, U.S. Pat. Nos. 5,668,110, 5,683,983, 5,667,280 and 5,654,276 disclose the use of formoterol in combination with IL-5 inhibitors, U.S. Pat. No. 6,136,603 discloses the use of formoterol in combination with antisense modulators of IL-5, U.S. Pat. No. 5,602,110 discloses the use of formoterol in combination with millrinone, U.S. Pat. No. 5,525,623 discloses the use of formoterol in combination with a tryptase inhibitor, U.S. Pat. Nos. 5,691,336, 5,877,191, 5,929,094, 5,750,549 and 5,780,467 disclose the use of formoterol in combination with a tachykinin receptor antagonist, International PCT Application Publication No. WO 99/00134 discloses the use of formoterol in combination with rofleponide and International PCT Application Publication No. WO 99/36095 discloses the use of formoterol in combination with a dopamine D₂ receptor agonist. The present invention includes the use of such combinations.

In accordance with an embodiment, the method of the present invention is used to administer formoterol in combination with one or more of (a), (b), (c) or (d) as follows: (a) a β₂-adrenoreceptor agonist; (b) a dopamine (D₂) receptor agonist; (c) a prophylactic therapeutic, such as a steroid; or (d) an anticholinergic agent; which is administered simultaneously with, prior to or subsequent to the formoterol-containing solution.

In accordance with a specific embodiment of the present invention, the solution for administration includes budesonide, sodium chromoglycate, and/or nedocramil sodium salts in combination with formoterol.

The micronized powder formulation for use according to the present invention comprises (A) the active ingredient, formoterol, alone or in combination with at least one other active ingredient, and (B) a finely divided pharmaceutically acceptable carrier, which may be one or more materials chosen from materials known as carriers in dry powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran or mannitol. An especially preferred carrier is lactose, particularly in the form of the monohydrate. The dry powder can be stored in capsules of gelatin or plastic, or in blisters, as for use in a dry powder inhalation device, preferably in dosage units to bring the total weight of powder in each capsule or blister to from 5 mg to 50 mg. Alternatively, the dry powder can be contained in a standard vial or other container suitable for dry storage of a pharmaceutical powder formulation.

In order to maintain stability of the formoterol in the micronized powder formulation, the formulation is maintained free, or substantially free, of water prior to use in the method of the present invention.

In the finely divided particulate form of the powder formulation, the active ingredient(s) has an average particle diameter of up to about 10 μm, for example 0.1 to 5 μm, preferably 1 to 5 μm. The solid carrier, where present, generally has a maximum particle diameter of about 300 μm, preferably about 212 μm, and conveniently has a mean particle diameter of 40 to 100 μm, preferably 50 to 75 μm. The particle size of the active ingredient(s) and that of a solid carrier, where present, in dry powder compositions, can be reduced to the desired level by conventional methods, for example by grinding in an air-jet mill, ball mill or vibrator mill, microprecipitation, spray-drying, lyophilisation or recrystallisation from supercritical media.

The ingredients of the powder formulation can be formulated by the following examples which are not intended to limit the scope of the invention:

1. Mixing 4.5 parts by weight of formoterol fumarate dihydrate with 915 parts by weight of lactose monohydrate. Micronizing the resulting blend using a high pressure air jet mill and then conditioned using the process of EP-A-717 616.80.

2. Mixing 12 parts by weight of formoterol fumarate with 788 parts by weight of lactose monohydrate. Micronizing the resulting blend using a high pressure air jet mill and then conditioned using the process of EP-A-717 616,200.

Micronization of the blend of formulation ingredients can be carried out in a conventional manner such that the particle size range is suitable for inhalation. The particle size is preferably less than 25 μm, more preferably less than 10 μm and most preferably approximately 5 μm.

### Method of Administration

The method of the present invention includes the step of dissolving the powder formulation described above in a pharmaceutically acceptable solvent. The solvent can be, but is not restricted to, water or saline. The selection of a suitable solvent for preparation of the nebulizer solution would be routine for a worker of ordinary skill in administration of medicine via a nebulizer.

As described in more detail above, the formoterol can be administered in combination with one or more additional active ingredients, which is/are administered simultaneously with, prior to or subsequent to formoterol administration. In the embodiment in which the one or more additional active ingredient is administered simultaneously with formoterol, the one or more additional active ingredients can be in the powder formulation or in the solvent. In accordance with a specific embodiment of the present invention, the solvent is a ipratropium bromide solution or a budesonide solution, which is used to dissolve the formoterol containing powder formulation.

Following the dissolution of the powder formulation in a suitable solvent the resulting solution is administered to the patient within 30 minutes following the dissolving step. In accordance with a specific embodiment of the invention, the solution is administered within 15 minutes following the dissolving step.

The formoterol-containing solution can be prepared in a separate container or receptacle and the appropriate amount required to deliver the desired dose of formoterol, and the one or more additional active ingredient if present, is transferred to the nebulizer receptacle. The term “nebulizer receptacle” as used herein, refers to that portion of a nebulizing device that comprises a container or a reservoir for the solution to be nebulized. Alternatively, the formoterol-containing solution can be prepared directly in the nebulizer receptacle.

The magnitude of a prophylactic or therapeutic dose of formoterol in the acute or chronic management of disease will vary with the severity of the condition to be treated. The dose, and perhaps the dose frequency, will also vary according to the age, body weight, and response of the individual patient. Further, it is noted that the clinician or treating physician would know how and when to interrupt, adjust, or terminate therapy in conjunction with individual patient response.

In accordance a specific embodiment of the present invention, formoterol is administered in individual unit doses from 4.5 μg up to 12 μg. In accordance another embodiment of the invention, the steps of dissolving the powder formulation to prepare the formoterol-containing solution and administering the solution are repeated in a maintenance therapy. For example, administration can be twice daily for maintenance therapy with a suitable unit dose of formoterol (e.g., as formoterol fumarate dihydrate) in the range of from 1 μg to 40 μg and more preferably between 4.5 μg up to 12 μg. The daily dose of formoterol (as formoterol fumarate dihydrate) including maintenance therapy should be in the range of from 1 μg to 100 μg, preferably from 2 μg to 40 μg and more preferably from 3 μg to 40 μg. Additional dosing may be up to every 4 hours for breakthrough symptoms up to a daily maximum dosage of 80 μg, for patients sent home.

The formoterol powder formulation can be in the form of unit doses in a blister pack having a variable number of doses prepared from methods known to those skilled in the art. Alternate dispensing means include pull-apart capsules prepared from methods known to those skilled in the art. The unit dose of formoterol dissolves instantly in 2 to 3 ml of various solvents including, but not restricted to, water, normal saline, an ipotropium bromide solution, and budesonide solution to form a solution with no particles that is suitable for administration via nebulization. Nebulization occurs with the same rapidity as with other nebulized products and the duration of action is as documented for formoterol administration using other delivery devices. No residue is left in the nebulizer receptacle confirming that the total dosage is nebulized.

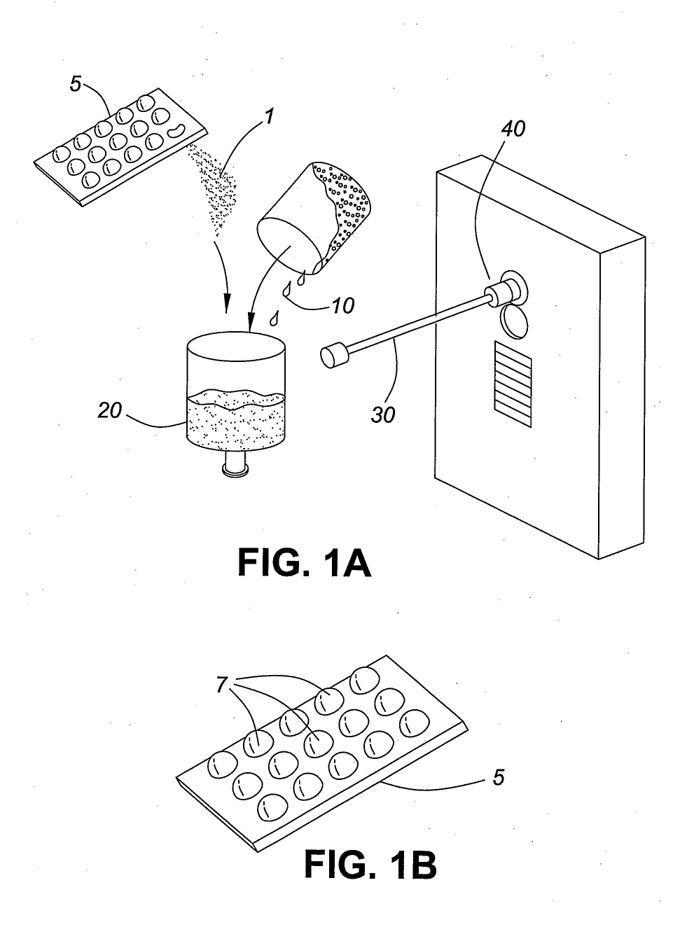

The nebulizer is an instrument that is capable of generating very fine droplets for inhalation. Within this device the nebulized liquid or solution is atomized into a mist of droplets of varying sizes by atomization means including, but not limited to, compressed air, ultrasonic waves or a vibrating orifice. Specific means suitable for atomizing a solution or suspension in accordance with the method of the present invention include compressed air or oxygen delivered by a wall outlet in a hospital emergency room (see FIGS. 1, 2 and 3).

Nebulizers containing no propellant and suitable for use in the method of the present invention include, but are not limited to, jet nebulizers (optionally sold with compressors), ultrasonic nebulizers and others. The nebulizer can be an oral nebulizer or a nasal nebulizer (e.g., U.S. Pat. Nos. 5,906,198 and 6,962,151). Nebulizers are available from, e.g., Pari GmbH (Starnberg, Germany), DeVilbiss Healthcare (Heston, Middlesex, U.K.), Healthdyne, Vital Signs, Baxter, Allied Health Care, Invacare, Hudsin, Omron, Bremed, Airsep, Luminscope, Medisana, Siemens, Aerogen, Mountain Medical Ltd. (Colchester, Essex, U.K.), AFP Medical (Rugby, Warwickshire, U.K.) Bard Ltd. (Sunderland, U.K.), Carri-Med. Ltd. (Dorking, U.K.) and many others.

Exemplary jet-nebulizers for use in the method of the present invention include but are not confined to: Pari LC plus/ProNeb, Pari LC plus/ProNeb Turbo, Pari LC plus/Dura Neb 1000 & 2000, Pari LC plus/Walkhaler, Pari LC plus/Pari Master, Pari LC star. Omron Compair XL, Portable Nebulizer System (NE-C18 and JetAir disposable nebulizer), Omron CompAir Elite Compressor Nebulizer System (NE-C21 and Elite Air Reusable Nebulizer), Pari LC Plus or Pari LC Star nebulizer with Proneb Ultra compressor, Pulmo-aide, Pulmo-aideLT, Pulmo-aide traveler, Invacare Passport, Inspiration Healthdyne 626, Pulmo-Neb Traveler, DeVilbliss 646, Whisper Jet, Acorn 11, Misty-Neb, Allied aerosol, Schuco Home Care, Lexan Plastic Pocet Neb, SideStream Hand Held Neb, Mobil Mist, Up-Draft Up-Draft 11, T-Up-Draft, ISO-NEB, AVA-NEB, Micro Mist and PulmoMate.

Exemplary ultrasonic nebulizers for use in the method of the present invention include, but are not confined to, MicroAir, UltraAir, Siemens Ultra Nebulizer 145, CompAir, Pulmosonic, Scout, 5003 Ultrasonic Neb., 5004 Desk Ultrasonic Nebulizer, Mystique Ultrasonic, Luminscope Ultrasonic Nebulizer, Medisana Ultrasonic Nebulizer, Microstat Ultrasonic Nebulizer and MABIS Mist Hand Held Ultrasonic Nebulizer.

Other nebulizers for use herein include 5000 Electromagnetic Neb, 5001 Electromagnetic Neb, 5002 Rotary Piston Neb, Lumineb 1 Piston Nebulizer, Aeroneb Portable Nebulizer System, Aerodose inhaler, and AeroEclipse Breath Actuated Nebulizer.

The dissolving step of a specific embodiment of the method of the present invention is depicted in FIG. 1A, in which a unit dose of the formoterol-containing powder formulation 1 is dispensed from a blister pack 5 and mixed with normal saline 10 as the diluent directly in the nebulizer receptacle 20. Plastic tubing 30 connects nebulizer receptacle 20 to a wall oxygen/air flow meter and outlet 40. FIG. 1B is a detailed cross-sectional view of blister pack 5 showing individual unit dose blisters 7, each containing a unit dose of a formoterol-containing powder formulation.

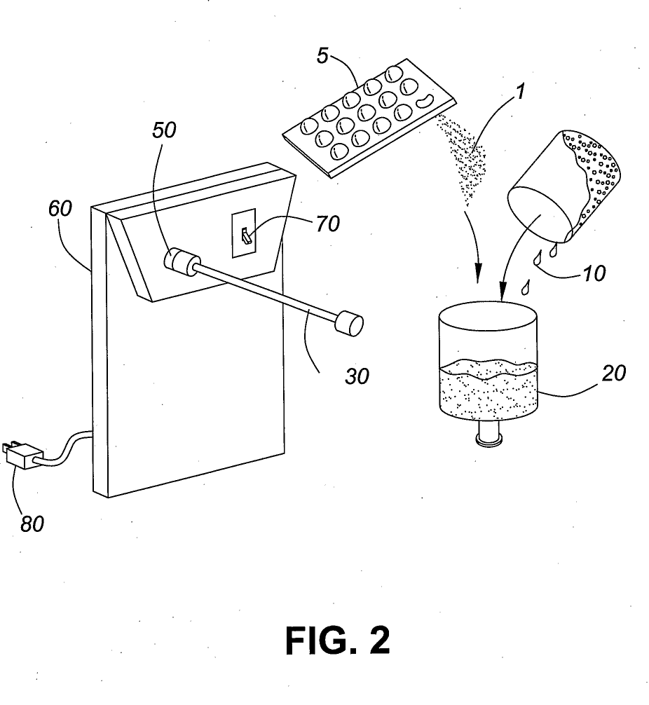

The dissolving step of another specific embodiment of the method of the present invention is depicted in FIG. 2, in which a unit dose of the formoterol-containing powder formulation 1 is dispensed from a blister pack 5 and mixed with normal saline 10 as the diluent directly in the nebulizer receptacle 20. Plastic tubing 30 connects nebulizer receptacle 20 to the nozzle 50 for compressed air of a home nebulizer 60, having an on/off switch 70 and electrical cord and plug 80 for inserting in a wall socket as a power source.

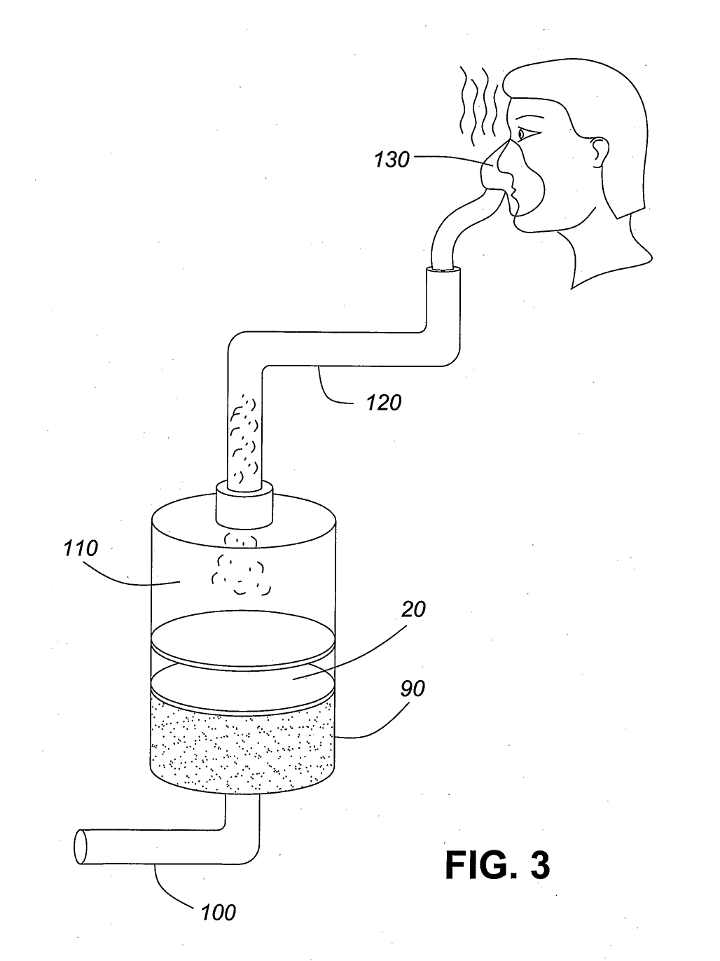

The administering step of a specific embodiment of the method of the present invention is depicted in FIG. 3, in which the formoterol-containing nebulizer solution 90 is aerosolized in the nebulizer receptacle 20 using compressed oxygen/air provided from a wall unit or nebulizer via plastic tubing 100. The aerosolized formoterol solution 110 travels through the administration plastic tubing 120 to a face mask 130 worn by the patient who breathes in the aerosolized formoterol solution.

### Applications

The method of the present invention is used for the treatment, prevention or amelioration of one or more symptoms of bronchoconstrictive disorders in a patient in need thereof. Bronchoconstrictive disorders affect millions worldwide. Such disorders include asthma (including bronchial asthma, allergic asthma, exercise induced asthma and intrinsic asthma, e.g., late asthma and airway hyper-responsiveness), chronic bronchitis and other chronic obstructive pulmonary diseases. The method of the present invention can also be performed using a nasal nebulizer to treat, prevent or ameliorate nasal inflammatory disorders such as allergic rhinits.

In accordance with specific embodiment of the present invention, the method is used to treat, prevent or ameliorate one or more of the following conditions:
acute condition of asthma (i.e. acute asthma attacks);
intermittent asthma;
short episodes of acute attacks of bronchospasm in chronic asthma; and/or
other respiratory illnesses with bronchospasm (e.g., COPD)

It has now been found that the method of the present invention is useful for treating patients of all age groups, irrespective of handicaps or mental state.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A method for the treatment, prevention, or amelioration of one or more symptoms of bronchoconstrictive disorders in a patient, comprising the steps of:
(a) providing a micronized powder formulation comprising formoterol, or a pharmaceutically acceptable salt or adduct thereof, and a pharmaceutically acceptable excipient;
(b) dissolving said powder formulation in a solvent to form a solution; and
(c) administering an amount of said solution to said patient via a nebulizer within 30 minutes following the dissolving step.

2. The method according to claim 1, wherein said formoterol, or a pharmaceutically acceptable salt or adduct thereof, is formoterol fumarate.

3. The method according to claim 2, wherein said formoterol, or a pharmaceutically acceptable salt or adduct thereof, is formoterol fumarate dihydrate.

4. The method according to claim 1, wherein said solvent is saline or water.

5. The method according to claim 1, wherein said solution is administered to said patient via a nebulizer within 15 minutes following the dissolving step.

6. The method according to claim 1, wherein said excipient is lactose monohydrate.

7. The method according to claim 1, wherein said powder formulation comprises approximately 4.5 parts by weight of formoterol fumarate dihydrate mixed with approximately 915.5 parts by weight of lactose monohydrate.

8. The method according to claim 1, wherein said powder formulation comprises approximately 12 parts by weight of formoterol fumarate dihydrate mixed with 788 parts by weight of lactose monohydrate.

9. The method according to claim 1, wherein steps (a), (b) and (c) are repeated to administer a maintenance therapy of two doses per day of said formoterol to said patient.

10. The method according to claim 1, wherein a dose of between about 1 μg to about 40 μg of formoterol is administered to said patient.

11. The method according to claim 1, wherein a dose of between about 4.5 μg to about 12 μg of formoterol is administered to said patient.

12. The method according to claim 1, wherein said solution comprises one or more active ingredients in addition to said formoterol, or pharmaceutically acceptable salt or adduct thereof.

13. The method according to claim 1, wherein said patient has a bronchoconstrictive disorder.

14. The method according to claim 13, wherein the bronchoconstrictive disorder is asthma chronic bronchitis, or a chronic obstructive pulmonary diseases.

15. A kit for performing the method according to claim 1, comprising:
(a) one or more containers of said powder formulation comprising said formoterol, or a pharmaceutically acceptable salt or adduct thereof, and a pharmaceutically acceptable excipient; and
(b) instructions for use.

16. The kit according to claim 15, which additionally comprises one or more containers of said solvent.

17.-29. (canceled)
